(19) United States
(12) Patent Application Publication
(10) Pub. No.: US 2012/0122199 A1
(43) Pub. Date: May 17, 2012

(54) Photobioreactor for the growth and development of photosynthetic and heterotrophic microorganisms

(76) Inventor: Edouard Kabakian, Gaillac (FR)

(21) Appl. No.: 13/376,942

(22) PCT Filed: Jun. 9, 2010
(86) PCT No.: PCT/FR10/00420

§ 371 Date: Jan. 27, 2012

(30) Foreign Application Priority Data
Jun. 9, 2009 (FR) 0902781

(51) Int. Cl. C12M 1/42

(52) U.S. Cl. 435/292.1

(57) ABSTRACT

A photobioreactor, especially for the growth and development of photosynthetic microorganism such as microalgae or cyanobacteria. The photobioreactor includes at least one reflector (101) placed on one side of the photobioreactor and tubes arranged in a plurality of layers following one after another along a direction (N) normal to the reflector taken at a central region thereof, each layer including a plurality of tubes. Preferably, the tubes (1, 16, etc.) are straight and extend along a direction orthogonal to the normal direction (N), the reflector is cylindrical of circular or oval cross section, and the layers are cylindrical, of circular or oval cross section, and concentric.

The present invention relates to a photobioreactor, especially for the growth and development of photosynthetic and heterotrophic microorganisms such as microalgae or cyanobacteria.

Microalgae and cyanobacteria are aquatic organisms varying in size from 1 micron to around 100 microns and use light as energy source to fix carbon dioxide (CO₂). Like terrestrial plants, microalgae and cyanobacteria are able to accumulate the absorbed carbon in the form of lipids, thereby making it possible to envisage using them to produce biofuels. Such a use is all the more promising in that microalgae and cyanobacteria have a very high photosynthesis yield and a very high cell growth rate (from ten to several tens of times higher than those of terrestrial oleaginates such as rapeseed, sunflower, etc.) and in that the fraction of biomass directly usable is maximal (conversely, terrestrial plants divert some of the absorbed carbon into lignocellulosic molecules, which are difficult or even impossible to utilize).

To maximize production of usable lipids (essentially glycerides), it is recommended to subject the microalgae and cyanobacteria to alternating growth and oil-production cycles. Growth is obtained by supplying the microalgae with carbon dioxide and nitrogen, under medium-to-low light intensity. Oil production is simulated by a stress generated by a nitrogen deficiency and/or a sudden increase in light intensity. When the conditions are optimized, the amount of lipids that the microalgae and cyanobacteria can accumulate is up to 80% of their dry weight.

Currently, there are two ways of producing microalgae and cyanobacteria: open-air culture, in ponds of the “race track” type, and culture in a transparent closed vessel, called a photobioreactor. The open cultures offer lower yields, require a large influx of water to compensate for evaporation, and are sensitive to contamination. Photobioreactors may compensate for a higher cost by high productivity levels by virtue of greater control of the conditions of access to the nutritient resources, exposure to light and transfer of CO₂ from the gas phase to the liquid phase.

There are two broad types of photobioreactor: flat photobioreactors and tubular photobioreactors.

Flat photobioreactors are essentially composed of two parallel transparent panels between which a thin layer of culture medium flows along a path with baffles. Flat photobioreactors are currently out of favor because of leakage problems that they encounter, because of their propensity to soiling (due to the baffles) and because of the large number of units it would be necessary to employ in order to envisage operation on an industrial and commercial scale.

Tube photobioreactors comprise one or more transparent tubes of various lengths and diameters (or widths). The following may be distinguished:
column-type photobioreactors, formed from a wide vertically standing column, the diameter of which generally varies between 30 and 60 cm;
planar photobioreactors that include a plurality of rigid tubes, generally of smaller diameters (less than 15 cm), which are placed by side by side and connected so as to form a coil, the tubes all lying in one and same horizontal, inclined or vertical plane;
triangular photobioreactors comprising a plurality of triangular tubes placed side by side, the photobioreactor consequently having the form of a prism with a triangular base;
helical photobioreactors, formed using a single tube of great length wound in a helix around a vertical structure; and
photobioreactors in which the tubes are formed in a rigid extruded double-walled panel.

The exposure of the microorganisms to light depends on the geometry of the photobioreactor. Tubular photobioreactors permit great flexibility in terms of size and volume and can be easily fitted with stirring and circulation devices of various types, depending on the microalgae or cyanobacteria cultivated.

This being so, the known tubular photobioreactors have the drawback of a large footprint, thereby limiting the efficiency per hectare obtained.

The aim of the invention is to alleviate this drawback by proposing a tubular photobioreactor of innovative geometry, the footprint of which is reduced, for a yield equivalent to or greater than that provided by a known photobioreactor of the same culture volume.

To do this, the invention proposes a photobioreactor comprising a plurality of reaction tubes, which is distinguished by the fact that it comprises at least one reflector placed on one side of the photobioreactor and by the fact that the tubes are arranged as a plurality of layers following one another along a direction normal to the reflector taken in a central region thereof, each layer comprising a plurality of tubes.

Preferably, the tubes are arranged in the photobioreactor so that each tube is not completely obscured by another tube along this normal direction.

The photobioreactor according to the invention is intended to be installed outdoors so that the reflector extends beneath the tubes and reflects the sun's rays onto those tubes. Preferably, it is installed so that the normal direction defined above coincides substantially with the zenithal direction. The invention is therefore based on the combined use of a reflector and of the vertical superposition of layers of tubes. The photobioreactor according to the invention has as small footprint compared to known tubular (triangular or planar) photobioreactors.

Preferably, all the tubes of the photobioreactor according to the invention are connected in series to one another. As a variant, all the tubes are connected in parallel. As a variant, the photobioreactor comprises several groups of tubes, the tubes of any one group being connected in series and the groups being connected in parallel.

Advantageously, all the tubes of the photobioreactor according to the invention are straight and extend along the same direction orthogonal to the direction normal to the reflector. The tubes therefore extend horizontally when the photobioreactor is installed so that the direction normal to the reflector coincides substantially with the zenithal direction.

Advantageously, the reflector is cylindrical with circularly arcuate or oval (for example parabolic or semielliptical) cross section and the tubes extend parallel to the generatrix of the reflector. Thus, each tube receives substantially the same light intensity over its entire length when the photobioreactor is installed so that the tubes extend along a north-south direction or in north-south planes.

According to a preferred arrangement, the layers are all cylindrical, of circular or oval cross section, and concentric. The layers thus “follow” the course of the sun.

Advantageously, the photobioreactor according to the invention has at least two tubes of different diameter that are connected together. Since the flow rate of the culture medium is constant from one tube to another within the photobioreactor, the speed of flow is increased in the tube or tubes of smaller diameter. This acceleration of the culture medium prevents any sedimentation and flocculation of the microorganisms.

Preferably, the tubes of one and the same layer have identical diameters and the diameter of the tubes decreases from one layer to the next going from the periphery toward the center of the photobioreactor. It should be noted that the term “diameter” denotes here in general the largest transverse dimension of each tube (this is a diameter in the usual meaning of the term only when the tube has a right circular cross section, it being pointed out that the invention is not limited to tubes of circular cross section). Such an arrangement makes it possible not only to accelerate the culture medium in the tubes extending in the central portion of the photobioreactor, but also to optimize the exposure of the tubes to the light. This is because the quantity and the quality of the light actually available for each microorganism in a tube depends not only on the incident light flux striking the tube but also on the diameter of said tube: the irradiance is attenuated exponentially inside the tube as a function of the depth of the culture medium (for a given cell concentration) owing to a phenomenon known as “self-shading”, which is the result of light being absorbed and scattered by the microorganisms. In the photobioreactor according to the invention, the tubes located in the central portion of the photobioreactor are liable to be partially hidden along the direction of the incident light rays, by the tubes located in the peripheral portion of the photobioreactor. They therefore receive an incident light flux which, depending on the time of day, may be less than that striking the peripheral tubes. To compensate for this lesser exposure, the central tubes advantageously have a smaller diameter, which limits energy losses by self-shading within the tube. Preferably, the difference in diameter between the peripheral and central tubes is chosen so as not to completely compensate for the difference in exposure of said tubes, in order to create zones of lower irradiance and zones of higher irradiance. A sudden increase in the irradiance can actually stimulate the production of lipids within the microorganisms. However, choosing to adjust the diameters of the peripheral and central tubes so as to obtain an irradiance which is substantially linearly uniform (along the tubes) in the overall photobioreactor is not ruled out.

Advantageously, for each layer, the number of tubes and the diameter thereof are chosen so that the tubes occupy between 35% and 50% of the area of the layer (the layer passing through the central axes of the tubes). In other words, 50% to 65% of the incident light rays pass from one layer to the next layer running from the periphery toward the center of the photobioreactor. Such an arrangement makes it possible to optimize both the footprint of the photobioreactor and exposure to the sun of the tubes located in the central portion of the photobioreactor.

Advantageously, the photobioreactor according to the invention comprises at least one device, preferably a pump or possibly a gas injector, for the axial flow of a culture medium inside each tube. Moreover, the photobioreactor includes at least one, preferably helical, deflector for each tube, which deflectors promote the mixing and homogenization of the culture medium. It is possible to replace the pump and the deflectors (which are stationary) by stirring means, of the propeller type, turbine type, etc., in a plurality of the tubes or in each of them, provided that these means are not of a nature to harm the microorganisms. The use of deflectors has the advantage of maintaining cell integrity.

Preferably, the tubes are connected in series by return bends on the two opposed end faces of the photobioreactor. Advantageously, the return bends are placed so that at most three (or possibly four) tubes of any one layer are consecutive. In other words, the culture medium passes several times from one layer to another during one complete cycle. It is thus subjected to different amounts of sun exposure, flows at different speeds, etc., in an alternating manner.

Also preferably, the return bends are placed so that the path traveled by the flowing culture medium is as horizontal as possible when the photobioreactor is installed so that the direction normal to the reflector corresponds substantially with the zenithal direction and/or so that the tubes extend horizontally.

In order better to control the synthesis taking place in the tubes of the photobioreactor according to the invention, it is proposed in one embodiment that at least certain tubes each be equipped with at least one diffuser enabling products to be injected into said tubes of the photobioreactor. As an example, it is thus possible to inject into the photobioreactor CO₂, NOx, nutrients, organic carbon, etc.

In order better to control synthesis in the photobioreactor, at least one artificial lighting means for illuminating the reaction tubes is advantageously provided. This therefore makes it possible to illuminate the tubes of the photobioreactor when there is no sunshine or when there is insufficient light during the day.

In the advantageous embodiment in which the tubes are arranged in concentric layers, the artificial lighting is preferably placed at the center of the layers for greater efficiency.

A preferred embodiment provides for the reflector to be a wavelength-selective reflector, that is to say one that reflects light in a range of wavelengths and allowing light outside said range of wavelengths to be passed there through. In such a case, light energy passing through the reflector is advantageously recovered by at least one photovoltaic sensor placed beneath the reflector. The energy thus recovered at the photovoltaic sensors can then be used to supply the artificial lighting with energy.

Finally, provision may be made for the photobioreactor to further include a protective cover, which may for example be used for thermal protection at night. Other details and advantages of the present invention will become apparent on reading the following description, which refers to the appended schematic drawings and relates to preferred embodiments provided as nonlimiting examples. In these drawings:

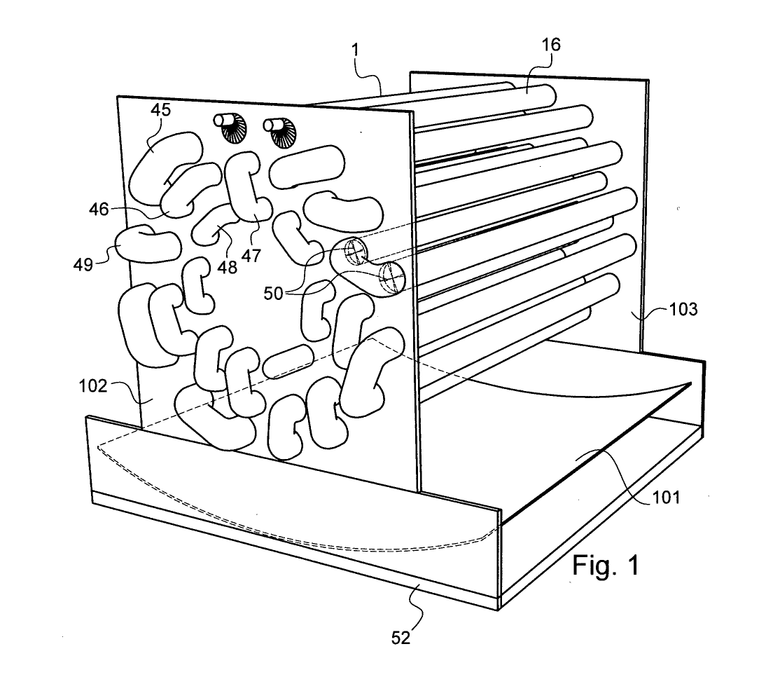

FIG. 1 is a schematic view of a main part of a photobioreactor according to the invention; and

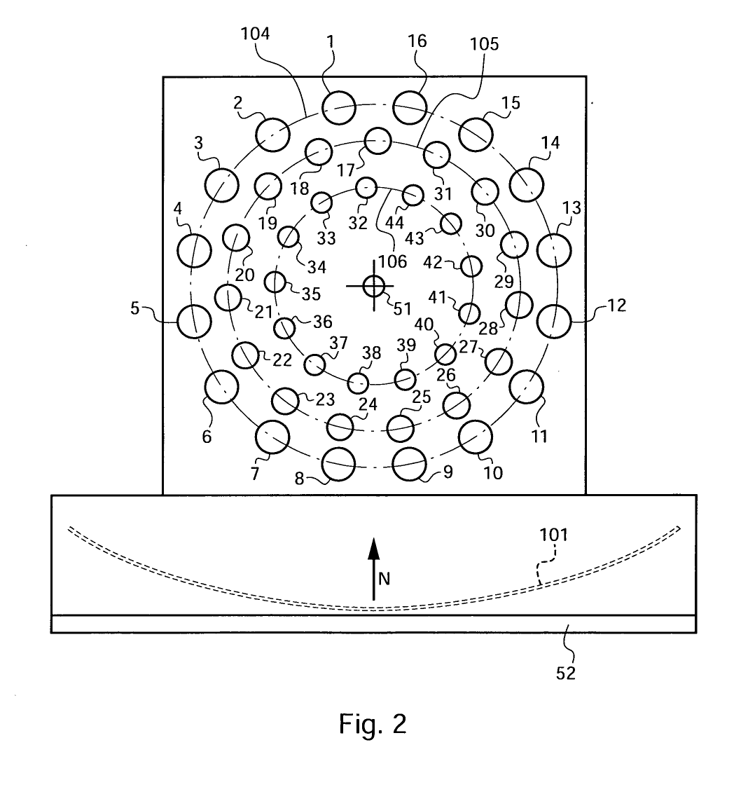

FIG. 2 is a schematic cross-sectional view of the tubes and of the reflector of the photobioreactor of FIG. 1.

The photobioreactor according to the invention illustrated in FIGS. 1 and 2 is shown in a position corresponding to its use position. The terms “vertical”, “horizontal', “above”, “beneath”, “lower”, “upper”, etc., refer to this position.

The photobioreactor illustrated comprises a lower reflector 101 intended to be directed toward the sun. This reflector is cylindrical, of parabolic or circularly arcuate cross section. Lying above this reflector is a plurality of cylindrical tubes 1 to 44. All the tubes are parallel to the generatrix of the reflector 101. Each tube 1 to 44 measures about 6 meters in length and the tubes lie transversely one with respect to another. Each tube 1 to 44 is supported at each of its ends by a support plate 102, 103. Just these two plates serve for fastening and supporting all the tubes. Each tube 1 to 44 has a right circular cross section, so as to limit any deposits liable to form on the internal wall of the tube. Each tube 1 to 44 is made of a plastic, such as a polycarbonate, which is preferably non-stick or provided with an internal non-stick coating so as to prevent or limit the formation of deposits on the internal wall of the tube.

The reference N indicates a direction normal to the reflector 101 contained within a longitudinal median plane thereof (the term “median” meaning that the plane cuts the reflector into two equal portions). According to the invention, the tubes are arranged in layers one after another along this normal direction above the reflector 101. In the example illustrated, the photobioreactor comprises three cylindrical layers of circular cross section. These layers are moreover concentric, their common center being about 3 meters above the ground.

Such a configuration makes it possible to house a maximum number of tubes per unit area of ground, while still guaranteeing optimum irradiance for each of the tubes.

More precisely, the photobioreactor illustrated comprises:
an outer cylindrical layer 104 (of circular cross section) of radius R1 of about 182 cm, this layer 104 comprising 16 tubes referenced 1 to 16, all having an inside diameter of about 34 cm;
an intermediate cylindrical layer 105 (of circular cross section) of radius R2 of about 142 cm, this layer 105 comprising 15 tubes referenced 17 to 31, all having an inside diameter of around 28 cm; and
an inner cylindrical layer 106 (of circular cross section) of radius R3 of about 92 cm, this layer 106 comprising 13 tubes referenced 32 to 44, all having an inside diameter of around 22 cm.

The tubes of the intermediate layer 105 are angularly offset relative to the tubes of the outer layer 104 with an offset angle chosen so as to maximize the amount of light reaching the tubes of the intermediate layer. For this purpose, the tube 17 is preferably centered angularly relative to the tubes 1 and 16. Likewise, the tubes of the inner layer 106 are angularly offset relative to the tubes of the outer layer 104 and intermediate layer 105 with an offset angle chosen so as to maximize the amount of light reaching the tubes of the inner layer. In the example illustrated, the tube 33 is angularly centered relative to the tubes 18 and 19. Moreover, the arrangement of all the tubes is chosen so that a significant portion of the incident light rays passes through all the layers and reaches the reflector 101 in order to reflect said rays toward the tubes.

In the configuration illustrated, the photobioreactor according to the invention has an area illuminated by the sun of about 235 m² and occupies a ground area of 35 m², i.e. a multiplicative factor of 6.7. Moreover, said photobioreactor may contain a culture medium volume of 17 210 liters.

In each circular layer, the tubes are separated along a circular arc by a distance equal to the diameter of the tubes of the layer increased by a multiplicative factor of between 1.01 and 1.15.

The tubes are connected in series by means of return bends 45-49 projecting from the support plates 102, 103 (toward the outside of the photobioreactor). For the sake of clarity, only the return bends located on the side having the support plate 102 are shown.

The return bends are arranged so as to make a culture medium flow along the following path: tube 1, tube 2, tube 3, tube 19, tube 18, tube 17, tube 32, tube 33, tube 34, tube 20, tube 4, tube 5, tube 6, tube 22, tube 21, tube 35, tube 36, tube 37, tube 23, tube 7, tube 8, tube 9, tube 25, tube 24, tube 38, tube 39, tube 40, tube 26, tube 10, tube 11, tube 12, tube 28, tube 27, tube 41, tube 42, tube 29, tube 13, tube 14, tube 30, tube 43, tube 44, tube 31, tube 15, tube 16. Advantageously, this path is defined so as to limit the vertical flow of the culture medium. Entry of the culture medium into the photobioreactor takes place at the top of the photobioreactor via the tube 1. The medium also exits therefrom at the top via the tube 16, to be filtered or reintroduced into the tube 1 for an additional cycle.

Consequently, the following return bends are provided on the side with the support plate 102: return bend 45 connecting the tubes 2 and 3; a return bend connecting the tubes 19 and 18; a return bend connecting the tubes 17 and 32; a return bend connecting the tubes 33 and 34; a return bend connecting the tubes 20 and 4; a return bend connecting the tubes 5 and 6; a return bend connecting the tubes 22 and 21; a return bend connecting the tubes 35 and 36; a return bend connecting the tubes 37 and 23; a return bend connecting the tubes 7 and 8; a return bend connecting the tubes 9 and 25; a return bend connecting the tubes 24 and 38; a return bend connecting the tubes 39 and 40; a return bend connecting the tubes 26 and 10; a return bend connecting the tubes 11 and 12; a return bend connecting the tubes 28 and 27; a return bend connecting the tubes 41 and 42; a return bend connecting the tubes 29 and 13; a return bend connecting the tubes 14 and 30; a return bend connecting the tubes 43 and 44; and a return bend connecting the tubes 31 and 15.

Following return bends are therefore provided on the side with the support plate 103: return bend connecting the tubes 3 and 19; a return bend connecting the tubes 18 and 17; a return bend connecting the tubes 32 and 33; a return bend connecting the tubes 34 and 20; a return bend connecting the tubes 4 and 5; a return bend connecting the tubes 6 and 22; a return bend connecting the tubes 21 and 35; a return bend connecting the tubes 36 and 37; a return bend connecting the tubes 23 and 7; a return bend connecting the tubes 8 and 9; a return bend connecting the tubes 25 and 24; a return bend connecting the tubes 38 and 39; a return bend connecting the tubes 40 and 26; a return bend connecting the tubes 10 and 11; a return bend connecting the tubes 12 and 28; a return bend connecting the tubes 27 and 41; a return bend connecting the tubes 42 and 29; a return bend connecting the tubes 13 and 14; a return bend connecting the tubes 30 and 43; a return bend connecting the tubes 44 and 31; and a return bend connecting the tubes 15 and 16.

In an alternative embodiment, the path in tubes may also be in the following order: tube 1, then tubes 6, 7, 5, 2, 41, 40, 3, 4, 8, 12, 13, 14, 15, 11, 9, 10, 16, 17, 18, 19, 24, 23, 20, 21, 22, 27, 26, 25, 30, 31, 32, 29, 28, 33, 34, 35, 36, 37, 38, 39, 42, 43 and finally tube 44 at the exit. The return bends are therefore arranged accordingly so as to allow the biomass to flow through the photobioreactor along this path.

Each tube is equipped with at least three variable-flow diffusers (not shown) placed in the support plate 102, 103 at the inlet (in the flow direction of the medium) of said tube, namely: a first diffuser capable of releasing CO₂ into the tube; a second diffuser capable of releasing nitrogen oxides (NOx) into the tube, for the purpose of causing nitrous stress, temporarily and opportunely; and a third diffuser for delivering nutrients (especially silica and oligo elements). Each tube may also be equipped with a fourth diffuser for delivering organic carbon if the culture medium contains microorganisms that are heterotrophic with respect to carbon. As a variant, only seven tubes are equipped with one or more (CO₂, NOx, nutrient or organic carbon) diffusers. A specific diffuser may also be provided for operating the photobioreactor in heterotrophic mode. Such a diffuser is for example provided with a rod, the length of which may be around 25 cm (this value being illustrative but nonlimiting), in order to convey nutrients to the central inlet of each tube.

The photobioreactor also includes a variable flow pump (not shown) for circulating the culture medium. Since the photobioreactor illustrated has a volume of about 17 m³, the pump is advantageously chosen so as to be able to provide a flow rate of between 17 and 105 m³/h, that is to say between 1 and 6 complete cycles per hour. The speed of flow of the medium varies by a factor of one to two between the tubes 1 and 16 of the outer layer 104 and the tubes 32 to 44 of the inner layer 106. It should be noted that the photobioreactor possibly has, if necessary, a plurality of pumps.

Each tube incorporates two helical deflectors 50 (in the form of blade impellers or, according to a variant not shown, in the form of grooves), one deflector being located at the inlet of the tube and the other located at the outlet thereof. As a variant, the deflectors may be incorporated in the return bends. The shape (angle of attack, pitch, length, diameter, rounded edge, etc.) and the constituent material of the deflectors are chosen so as not to harm the microorganisms. These deflectors are conducive to keeping the culture medium stirred.

Preferably, the photobioreactor also includes, at the outlet, a buffer tank designed to receive the culture medium exiting the tube 16. The volume of this buffer tank may be around 1000 liters and is preferably located at the same height as the outlet of the tube 16 so as to avoid any pressure drop that would be due to a difference in level. This buffer tank may also serve as a branching tank for connecting two photobioreactors together, whether in series or in parallel. Advantageously, top of the buffer tank is provided with a membrane permeable to oxygen but impermeable to carbon dioxide. Depending on the case, if necessary a pump may be provided in addition or as an alternative, to create a partial vacuum in the buffer tank so as to remove the oxygen released by the microorganisms.

Such a photobioreactor is intended to be installed in such a way that the normal direction N coincides substantially with the zenithal direction and that the tubes 1 to 44 lie along north-south (horizontal) directions so as to capture the maximum amount of light energy throughout the course of the sun. It is also possible for the photobioreactor to be slightly inclined in order for the incident light rays to be orthogonal to the axes of the tubes in the middle of the day (when the sun is highest).

The photobioreactor according to the invention is particularly intended for the biofuel industry (for the development of lipid-rich microalgae), but also for the agrifoodstuffs industry and the cosmetic and pharmaceutical industry.

Under certain conditions, an artificial illuminating means 51 (FIG. 2) may be provided. In the preferred embodiment shown in the drawings, that is to say in the case when the layers of tubes are concentric circular layers, the illumination may be advantageously provided at the center of these layers. This artificial illumination means 51 preferably extends parallel to the tubes of the photobioreactor for optimally illuminating said tubes.

This artificial illuminating means 51 is in this case a centrifugal illumination means which substantially illuminates the least well illuminated zones. This artificial illuminating means 51 makes it possible, in addition (or as an alternative), to concentrate light in other zones, thus stimulating the photosynthetic microorganisms.

For a culture of heterotrophic algae, that is to say algae that depend on organic substances for their growth and feed, the artificial illumination means 51 may also be used to interrupt the nocturnal cycle and improve the fixing of organic matter in heterotrophic mode.

It is also proposed here for this artificial illumination means 51 to be supplied with energy. The reflector 101, in one embodiment, is a selective reflector which reflects the light in the range of wavelengths used by the microorganisms to photosynthesize and which lets the light outside this range of wavelengths through. The light therefore passing through the reflector 101 is then advantageously collected by a panel 52 of photovoltaic collectors placed beneath the reflector 101.

In general, light propitious to the microorganisms has a wavelength between 400 and 700 nm (10⁻⁹ m), which corresponds to about 45% of the light emitted by the sun. If the reflector is therefore transparent for the waves outside this wavelength range, 55% of the solar energy is therefore potentially available for the panel 52 and therefore for electricity generation.

The energy thus recovered may be used directly by the artificial illumination means 51 and therefore illuminate the tubes at an angle different from the angle of direct illumination by the sun or reflected by the reflector 101. In one embodiment, provision may also be made for the energy recovered by the panel 52 to be stored in a battery (not shown) and then to be used at will for illuminating the tubes.

To control the temperature in the photobioreactor better, it is proposed to provide it with a cover, preferably a plurality of covers.

For example, it may happen that the temperature at night, or even day temperature during certain days in winter, is too low for the microorganisms. In this case, the cover used is for example made of a transparent plastic that lets through the light rays useful for photosynthesis, while also having a heating and insulating power.

To limit thermal losses on cool or cold nights, a dark cover may be envisaged.

It is also possible to cover the photobioreactor with two covers. For example, in winter the photobioreactor may be covered during the day with a cover made of a transparent (greenhouse type) film, in order to maintain the temperature inside the system, and at night this cover be supplemented with a thicker cover.

However, on days when the temperature is too high, a ventilation system may be used to regulate the temperature. In the embodiments with a panel 52 of photovoltaic collectors, the ventilation system may also be supplied with electrical energy directly by the panel 52 or by means of batteries.

The photobioreactor is also preferably protected from UV (ultraviolet) radiation. A first means of protection may be obtained by the choice of materials for producing the tubes. A second means of protection proposed here is the photobioreactor to be protected by transparent UV-antireflective cover, this cover for example being arranged in the form of a circular arc over the photobioreactor and following the rotation of the sun.

The invention may be the subject of many variants of the embodiment illustrated, provided that these variants fall within the scope defined by the claims.

For example, the outer layer 104 could comprise seventeen tubes (34 cm in diameter) and have a radius of 192 cm.

As a variant, the photobioreactor could have the following features:
a first cylindrical layer of circular cross section, having a radius of around 200 cm and comprising twenty-five tubes of 22 cm inside diameter;
a second cylindrical layer of circular cross section, having a radius of around 160 cm and comprising twenty-five tubes of 18 cm inside diameter;
a third cylindrical layer of circular cross section, having a radius of around 124 cm and comprising twenty-five tubes of 14 cm inside diameter; and
a fourth cylindrical layer of circular cross section, having a radius of around 92 cm and comprising twenty-one tubes of 12 cm inside diameter.

This embodiment supplies 301 m² of illuminated area and occupies a ground area of 35 m², i.e. a multiplicative factor of 8.62, for a culture medium volume of 13 250 liters (if the length of the tubes is 6 m).

In high sunshine regions, it may be opportune to provide five consecutive rings, of 92 cm to 225 cm in radius, with tubes of 11 cm to 24 cm in diameter.

In general, the invention is not limited to the number of layers, to the radii of the layers, to the number of tubes, or diameter and length of the tubes that have been described and illustrated.

Various characteristics dimensions of the photobioreactor must be chosen especially according to the sunshine at the place of installation of the photobioreactor and the variety of microorganisms cultivated.

The invention is also not limited to layers of circular cross section. For example, it extends to planar layers.

1. A photobioreactor comprising a plurality of reaction tubes, which comprises at least one reflector (101) placed on one side of the photobioreactor and wherein the tubes are arranged in a plurality of layers (104-106) following one after another along a direction (N) normal to the reflector taken at a central region thereof, each layer (104, 105, 106) comprising a plurality of tubes (1-16, 17-31, 32-44).

2. The photobioreactor as claimed in claim 1, wherein all the tubes (1-44) are straight and extend along a direction orthogonal to the direction (N) normal to the reflector.

3. The photobioreactor as claimed in claim 1, wherein the reflector (101) is cylindrical, and of circularly arcuate or oval cross section, and wherein the tubes (1-44) extend parallel to the generatrix of the reflector.

4. The photobioreactor as claimed in claim 1, wherein the layers (104-106) are cylindrical, of circular or oval cross section, and concentric.

5. The photobioreactor as claimed in claim 1, wherein the tubes (1-16; 17-31; 32-44) of any one layer (104; 105; 106) have identical diameters and wherein the diameter of the tubes decreases from one layer to the next going from the periphery toward the center of the photobioreactor.

6. The photobioreactor as claimed in claim 1, wherein the diameter and the number of tubes (1-16, 17-31, 32-44) for each layer (104-106) are chosen so that the tubes occupy between 35% and 50% of the area of the layer.

7. The photobioreactor as claimed in claim 1, which includes at least one deflector (50) for each tube (1-44).

8. The photobioreactor as claimed in claim 1, wherein all the tubes (1-44) are connected in series to one another.

9. The photobioreactor as claimed in claim 1, wherein return bends (45-49) are placed between the tubes and so that at least three tubes of any one layer are consecutive.

10. The photobioreactor as claimed in claim 1, which comprises:
a cylindrical outer layer (104) of circular cross section, having a radius (R1) of about 182 cm and comprising sixteen tubes (1-16) all with an inside diameter of about 34 cm;
a cylindrical intermediate layer (105) of circular cross section, having a radius (R2) of about 142 cm and comprising fifteen tubes (17-31) all having an inside diameter of about 28 cm; and
a cylindrical inner layer (106) of circular cross section, having a radius (R3) of about 92 cm and comprising thirteen tubes (32-44) all having an inside diameter of about 22 cm.

11. The photobioreactor as claimed in claim 1, wherein at least certain tubes are each equipped with at least one diffuser for injecting products into said tubes of the photobioreactor.

12. The photobioreactor as claimed in claim 1, which includes at least one artificial illumination means (51) for illuminating the reaction tubes.

13. The photobioreactor as claimed in claim 12, which includes an artificial illumination means (51) placed at the center of the layers, and wherein the layers (104-106) are cylindrical, of circular or oval cross section, and concentric.

14. The photobioreactor as claimed in claim 12, wherein the reflector (101) is a wavelength-selective reflector, that is to say one reflecting light in the range of wavelengths and letting the light outside said range of wavelengths pass therethrough, said photobioreactor further including at least one photovoltaic collector (52) placed beneath the reflector (101).

15. The photobioreactor as claimed in claim 14, wherein the photovoltaic collectors (52) are used supplying the artificial illumination means (51) with energy.

16. The photobioreactor as claimed in claim 1, which further includes a protective cover.

17. The photobioreactor as claimed in claim 1, wherein the reflector (101) is a wavelength-selective reflector, that is to say one reflecting light in the range of wavelengths and letting the light outside said range of wavelengths pass therethrough, said photobioreactor further including at least one photovoltaic collector (52) placed beneath the reflector (101).
